(19) United States
(12) Patent Application Publication
(10) Pub. No.: US 2008/0308103 A1
(43) Pub. Date: Dec. 18, 2008

(54) Inhalator Capsules

(75) Inventors: Didier Lancesseur, Boulogne Billancourt (FR); Dieter Hochrainer, Schmallenberg (DE); Jorg Schiewe, Mainz (DE); Bernd Zierenberg, Bingen (DE)

Correspondence Address: SCOTT R. COX;LYNCH, COX, GILMAN & MAHAN, P.S.C., 500 WEST JEFFERSON STREET, SUITE 2100, LOUISVILLE, KY, 40202, US

(73) Assignees: AIRSEC S.A.S., Choisy le Roi (FR); BOEHRINGER INGELHEIM PHARMA GMBH & CO. KG, Ingelheim am Rhein (DE)

(21) Appl. No.: 11/914,463

(22) PCT Filed: May 17, 2006
(86) PCT No.: PCT/EP2006/004684

§ 371 Date: May 16, 2008

(30) Foreign Application Priority Data
May 18, 2005 (DE) 10 2005 022 862.3

(51) Int. Cl. A61M 16/00

(52) U.S. Cl. 128/203.21

(57) ABSTRACT

The subject matter of the invention is a capsule, in particular for the packaging of inhalation formulations, in which at least one cavity is enclosed by a wall, characterized in that at least one portion of the wall has a polymer composition that contains at least one adsorbent, as well as said capsule's preferred use. Furthermore an inhaler and secondary packaging containing at least one such capsule are also described.

The invention relates to a capsule as primary packaging, in particular for pharmaceutical inhalation formulations. The capsule according to the invention is an integral component of a ready-to-use powder inhaler.

### STATE OF THE ART

Medicinal aerosol therapy, directed at pulmonary inhalation, plays an important role in the treatment of numerous lung diseases. In addition to nebulizers for liquids containing active substances, powder inhalers in particular are used for the application of formulations of active substances in powder form.

In the area of powder inhalers, single-dose and multidose devices are known. In single-dose powder inhalers, dosing can be in the form of generally cylindrical capsules, which contain a powder formulation. Powder formulations contain the active substance in micronized form (with a particle size of approx. 1-5 μm), and generally one or more excipients. If a capsule is used as a container, it is opened in powder inhalers before the inhalation maneuver by piercing, crushing or cutting, so that the powder can be entrained from the capsule by the patient's breath, forming an airborne aerosol, which the patient inhales. Depending on the device, one or more capsules can be stored together in the device, or each capsule is inserted individually into the device at the time of use.

The capsules that are preferably used in a powder inhaler comprise two or more parts and are preferably of size 3. Such a powder inhaler is for example an inhaler of the HandiHaler® type, as disclosed for example in EP 1342483.

The manner in which the powder formulation for inhalation is packaged by the capsule is decisive for product quality and therefore suitability for use by inhalation. As a rule the inhalation powder comes into direct contact with the capsule material, so that the quality criteria for primary packaging have to be observed. Optionally, the primary packaging can be enclosed in a second outer protection, the secondary packaging, which must be removed before use. As a rule the secondary packaging encloses the primary packaging completely. Secondary packaging is used in particular when the primary packaging does not provide adequate protection for an unlimited time against e.g. moisture or other external factors. This secondary packaging can comprise e.g. film and foil containers made of aluminum foil (blisters etc.).

In such cases, as a rule the secondary packaging is removed first, before the sealed capsule is inserted in the powder inhaler. There, the capsule is then opened by an appropriate means of the powder inhaler.

The choice of a suitable material for the capsules is determined by two factors: on the one hand the material must be able to fulfill a certain protective function. On the other hand the material must be of such a kind that the capsule can be given the form required for use in the powder inhaler and can fulfill its proposed function. In the case of the HandiHaler® the capsule must be of such a kind that the powder can be discharged as a result of the Bernoulli effect that arises when the patient inhales.

Capsules for powder inhalers normally comprise hard gelatins, cellulose derivatives, starch, starch derivatives, chitosan etc., but capsules made from synthetic plastics, such as polyethylene, polycarbonate, polyester, polypropylene or polyethylene terephthalate etc. are also known.

The materials used until now have the disadvantage that they are permeable to moisture from the air in both directions. There is therefore a need to improve the ability of the capsules to keep the inhalation powder stable. Furthermore, materials such as gelatins are sensitive to moisture and may break if they are too dry. Conversely, in moist conditions they are sticky, which hampers efficient filling and/or use of the capsules, for example in inhalers. In particular, it may prove difficult to make an adequate or stable opening in the capsule for release of the powder. Some portion of the powder may still adhere to the capsule wall and thus not be available for the therapeutic effect after inhalation. To summarize, the mechanical and physicochemical properties of the products known in the state of the art, including products using the aforementioned plastics, are not completely satisfactory.

### DESCRIPTION OF THE INVENTION

One of the main functions of the capsule is to protect the active substance as well as the entire inhalation formulation against biological, chemical or physical alteration. Physical alteration includes in particular changes that can alter the discharge of the predetermined dose of fine particles. The term “dose of fine particles” means the dose that can reach the patient's lungs. This is influenced by the interactions of the micronized particles of active substance with one another as well as the interactions with the excipients. It has been found that especially as a result of a change in the moisture level inside the packaging, these interactions can increase to an extent that the dose of fine particles is greatly reduced. Such changes include penetration of water into the packaging and equally the removal of water from inside the packaging.

Therefore a main function of the packaging is to keep the chemical composition of the atmosphere inside the packaging constant, in order to avoid physical or chemical alteration of the formulation of active substance, or keep the inhalation formulation stable. In this connection, a distinction is made on the one hand between a short-term stability that the inhalation formulation must possess during the period of actual use (“in-use-stability”), i.e. the period from removal of the capsule from the additional protective packaging provided (secondary packaging), inserting in the inhaler, to opening of the capsule and inhalation of the inhalation powder and on the other hand the long-term stability, i.e. the stability that must be guaranteed for as long as the inhalation formulation is reliably protected by the unopened protective packaging (the secondary packaging). Another aim of the present invention was to provide a capsule which, while offering improved protection for the formulation of active substance, can be produced easily, has advantageous mechanical and physicochemical properties and avoids the drawbacks of the prior art.

This problem was solved by making capsules, such as those used e.g. in the HandiHaler®, from a material that comprises a polymer composition in which at least one adsorbent is incorporated.

It was found, surprisingly, that the manner of storage of powdered inhalation formulations can have a positive effect on the distribution of fine particles, and even the proportion of fine particles in the doses discharged can be increased. It was found that this can greatly prolong both the long-term stability of the pharmaceutical formulation as well as the in-use stability.

The present invention therefore relates to capsules that can be used together with a powder inhaler and as such are an integral component of the ready-to-use powder inhaler. The capsules can either be stored in the devices or they are inserted manually into the device at each use.

One function of the capsule and its material preferably comprises delaying and optionally minimizing the exchange of gaseous substances, in particular water vapor, between their interior and the surroundings.

Another problem comprises finding a capsule material that is suitable for making capsules preferably of size 3, which can be used in a Bernoulli-inhaler.

Another problem comprises producing capsules with a total mass and mechanical stability suitable for discharge from the inhaler. In this case a density of gelatine or polyethylene is advantageous.

### DETAILED DESCRIPTION OF THE INVENTION

The invention relates to a capsule, in particular for the packaging of inhalation formulations, in which at least one cavity is enclosed by a wall, characterized in that at least one part of the wall has a polymer composition that contains at least one adsorbent.

The shape of the container according to the invention depends on the intended use, e.g. the powder inhaler that is to be used. Preferably the container according to the invention is a two-part capsule, with the two parts fitting into one another telescopically, so that a capsule with an internal, enclosed cavity is formed. The inhalation formulation is contained in this cavity. Preferably the capsule according to the invention is of cylindrical shape with rounded ends. The pharmaceutical preparation cannot be discharged by the inhalation maneuver until the capsule has been opened in the inhaler. The capsule is preferably an integral component of a ready-to-use inhaler.

In this context the term “closed cavity” denotes a cavity enclosed by walls, from which a powder contained therein cannot escape without opening it.

The term “inhalation formulation” preferably denotes a pharmaceutical powder formulation, which preferably comprises readily nebulizing powder particles of active substance with a size (mean aerodynamic diameter) of less than 100 micrometers, preferably from 1 to 15 micrometers, more preferably from 1 to 5 micrometers. In addition, the pharmaceutical formulation can contain a readily flowing, pulverulent vehicle, e.g. lactose, with an average size <500 μm, preferably <200 μm, especially preferably <100 μm, and other excipients for improving the nebulizing property.

“Integral component of a ready-to-use inhaler” means that the capsule or an element is present in the inhaler, without which charging of the inhaler with the pharmaceutical formulation (inhalation formulation) for the purpose of inhalation is impossible or not provided. The capsule in the ready-to-use state can be connected rigidly to the inhaler, so that it cannot be removed nondestructively or without damaging the inhaler, or it is connected to the inhaler nondestructively, loosely or detachably.

Ready-to-use means that the container according to the invention is already inserted in the inhaler. This can have already been done at the factory, or the capsule is inserted in the inhaler by the patient. Optionally, the container is opened mechanically by components of the inhaler and/or is transported in the inhaler to the place of discharge. The order of these two steps can also be changed.

With the capsules according to the invention, an inhalation formulation is better shielded against the penetration of undesirable substances, in particular moisture from the surroundings, than is the case with comparable capsules known from the prior art.

The capsules according to the invention contain an adsorbent-polymer composition, i.e. at least one part of the capsule is made of such a material. According to a preferred embodiment of the invention the capsules comprise, completely or substantially, an adsorbent polymer composition, i.e. a polymer composition containing at least one adsorbent.

In the present description the expression adsorbent denotes a component that is capable of reacting or interacting with a chemical, undesirable component in the capsule (i.e. in the vicinity of the pharmaceutical composition), which has an affinity for the adsorbent, and can very probably be retained by the adsorbent. Although based on different reaction mechanisms, the expressions “adsorbent” and “absorbent” are used identically and interchangeably in the present description.

Preferably, any material can be used as adsorbent that is able to adsorb moisture or extract moisture from the surrounding atmosphere in some other way, or any material that is able to adsorb or otherwise extract other (undesirable) chemical components from the surrounding atmosphere, which also includes, without restriction, oxygen, carbon dioxide, carbon monoxide, amines, aldehydes, epoxides and alcohols.

In the present description and in the claims that follow, the expression “adsorbent” can also be used, according to a preferred embodiment, synonymously with the expression “drying agent” or “dehydrating agent”. Nonlimiting examples of suitable adsorbents comprise, among others, silica gels, drying clays or clays that adsorb moisture or water, aluminosilicates such as zeolites or bentonites, molecular sieves, activated carbon, alkaline-earth oxides, calcium sulfate or mixtures thereof.

According to a preferred embodiment of the invention, the adsorbent comprises at least one dehydrating agent. Nonlimiting examples of preferred dehydrating agents are silica gel, aluminosilicates such as bentonites or zeolites, molecular sieves and/or calcium sulfate.

The capsules according to the invention improve the usability of the filled capsules with respect to their functionality, their shelf life or storage stability, including pharmaceutical compositions contained therein. In particular they prevent negative effects of moisture or other harmful chemical components on the pharmaceutical compositions and the capsule material and any stickiness of the capsule walls due to increased moisture.

Furthermore, it was found, surprisingly, that the presence of at least one adsorbent or dehydrating agent in the polymer composition makes possible, in an advantageous manner, the piercing, cutting open or opening of the capsule wall in some other way, in particular by means of opening devices using pins or cutters, as used for example in existing powder inhalers, avoiding breaking apart or fragmentation of the capsules. Thus, the adsorbent also has an influence on the mechanical properties of the polymer composition in comparison with a polymer composition without adsorbent. This makes it possible, surprisingly, to provide capsules that have improved barrier properties, and at the same time have the required strength and penetrability for it to be possible to make an opening in the capsule wall e.g. by means of pins or cutting/opening devices, said opening being sufficiently large and stable for efficient release of the pulverulent pharmaceutical composition. It was also found, unexpectedly, that with the capsules according to the invention, the problem of partial or complete reclosing of the opening following its production by cutting or piercing the capsule wall with pins or cutting/opening devices, but before release of the powder from the capsule, is less pronounced or can even be avoided completely. It is assumed, though without the invention being limited to the accuracy of this assumption, that this positive effect can be attributed to the influence of the adsorbent on the mechanical and physical properties of the polymer composition. Thus, the capsules according to the invention combine stable, optimal protection of the powder composition with improved capacity for release of the pharmaceutical composition, including with other usual methods of opening the capsule, e.g. by separating the two parts of the capsule.

Preferably the adsorbent present in the polymer composition is a particulate adsorbent. The adsorbent can thus preferably be in the form of granules or powder. However, polymeric adsorbents can also be used within the scope of the invention.

According to an especially preferred embodiment of the invention, the polymer composition contains, based on the percentage by weight of the total composition, at least 5 wt. %, preferably at least 10 wt. %, in particular between about 10 and 50 wt. % of adsorbent. In general, with wall thicknesses greater than about 0.4 mm somewhat higher contents of adsorbent are preferably used, for example in the range from 10 to 80 wt. %. With thinner capsule walls, preferably of less than about 0.4 mm, the polymer composition preferably contains less than 50 wt. % adsorbent. It is of course possible to adjust the duration and degree of protection of the pharmaceutical composition packed in the capsule against harmful chemical components such as moisture by varying e.g. the amount of adsorbent in the polymer composition. Adsorption capacity and kinetics can also be adjusted on the basis of the type of adsorbent. Furthermore, its barrier function can be adjusted e.g. via the constitution of the polymer composition, other additives, the content of polymer, thickness of the capsule wall and the method used for production of the capsule walls. The specific areas for exchange between the external and the internal atmosphere of the capsule can also be adjusted.

According to a preferred embodiment of the invention, the polymer composition (polymer structure) used contains at least one thermoplastic material. Thermoplastic materials that can be used are generally all materials with thermoplastic properties, for example a polymer that was obtained from a single monomer, a copolymer from two or more monomers, a mixture of two or more polymers each from a single monomer, a mixture of two or more copolymers, or a mixture of at least one polymer, which was obtained from one monomer and at least one copolymer.

According to an especially preferred embodiment of the invention, the thermoplastic material used is polyethylene or polypropylene. According to another preferred embodiment of the invention, polystyrene can be used, especially when welding (e.g. ultrasonic welding) of the capsule parts is required. Polyamides may be preferred if for example especially thin capsule walls are required. To produce somewhat “softer” properties it is also possible to use silicones, for example.

Non-limiting examples of polymers (thermoplastic materials) from single monomers comprise: polystyrene, polyolefins, in particular polyethylene and polypropylene, polyacrylates, polymethacrylates, polyimides, polycarbonates, polyethersulfones, polyamides, polyesters and polyvinyl chlorides. Nonlimiting examples of copolymers comprise: ethylene-acrylate, modified polymers and maleic anhydride copolymers and graft copolymers. If a copolymer or a mixture is used, it is preferable to use a combination of monomers or polymers that have (at least) one chemical monomer unit in common. For example, a thermoplastic material can be used that contains linear low-density polyethylene (LLDPE), low-density polyethylene (LDPE) and ethylene-vinyl acetate copolymer (EVA), in which each of the components has ethylene as the monomer unit.

According to one possible embodiment of the present invention, the polymer composition contains no elastomer or only small amounts thereof. It was found that as a result, in many cases the phenomenon of reclosing of the opening made in the capsule wall for release of the pharmaceutical composition can be reduced quite significantly. According to an alternative embodiment, however, the polymer composition contains at least one elastomer, and once again the properties of the adsorbent-containing polymer composition are especially advantageous for the primary packaging and release of pulverulent pharmaceutical compositions. The elastomer can be selected, for example, from the group comprising styrene-butadiene rubber (SBR), styrene-ethylene-butadiene-styrene copolymers (SEBS), butyl rubber, ethylene-propylene rubber (EPR), ethylene-propylene-diene monomer rubber (EPDM), and ethylene-vinyl acetate copolymers (EVA) and butadiene-acrylonitriles.

Preferred materials are plastics that can be processed by injection molding or blow molding. Moreover, plastics that can be processed without needing a mold release agent, which can cause adherence of the filler on the wall, are advantageous. This has the advantage that there is no need to clean mold release agent from the interior of the container, in order for example to satisfy regulatory requirements restricting the use of mold release agents for primary packaging.

According to a preferred embodiment of the invention, the polymer composition can contain or comprise 5 to 85 wt. % of at least one polymer, in particular of at least one thermoplastic material, and 5 to 85 wt. % of at least one adsorbent.

The relative concentration of thermoplastic material and adsorbent can vary as a function of the thermoplastic material used and of the adsorbents used, preferably within the ranges stated previously. According to an especially preferred embodiment, the adsorbent polymer composition contains polyethylene, in particular high-density polyethylene (HDPE) and about 20 to 50 wt. %, in particular 20 to 40 wt. % of at least one particulate adsorbent or dehydrating agent.

The adsorbent preferably has a maximum particle size of less than 50 μm, in particular less than 40 μm, especially preferably less than 25 μm, measured in each case as max. 2% sieve residue. The average particle size, determined by the laser diffraction method (Malvern, according to the manufacturer's instructions, measurement in air) is between about 0.5 and 50 μm, preferably between 0.5 and 40 μm, in particular between 1 and 20 μm, and more preferably between 1 and 10 μm.

According to a preferred embodiment of the invention, the thickness of the capsule wall, at least in a section thereof, is between about 0.05 and 2 mm, in particular between about 0.1 and 1.1 mm, and more preferably between 0.1 and 0.5 mm. It was found that as a rule such a wall thickness provides both adequate stability of the formulation of active substance in the capsule according to the invention, as well as giving an advantageous capacity for release.

In addition it was found that, according to a preferred embodiment of the invention, the ratio of the maximum particle size of the adsorbent to the thickness of the capsule wall should be between 0.01 and 0.2, in particular between 0.02 and 0.1, as this gave especially advantageous stability and very good capacity for release of the formulations of active substance.

According to a preferred embodiment of the invention, the base mixture is prepared conventionally with the at least one polymer and the at least one adsorbent, by heating the polymer (thermoplastic material), to produce a melt, adding the adsorbent (if not yet done) and other conventional additives and mixing them in, after which the mixture can be converted to granules and stored in this form. Said granules can then be used for production of the desired structure (capsule), for example by extrusion forming, but in particular by injection molding.

According to a preferred embodiment of the invention, the at least one adsorbent is distributed uniformly over the thickness of the capsule wall or of the part thereof made from the polymer composition with at least one adsorbent. In this embodiment the polymer composition can be regarded as monolithic.

According to another preferred embodiment of the present invention, however, the at least one adsorbent is present at a higher concentration near the surface of the capsule wall or polymer composition than in its interior. Accordingly, the polymer composition used has at least one migration zone on one surface of the capsule wall and an inner region, with the maximum concentration of the at least one adsorbent within the migration zone being at least twice as high as the maximum concentration of the at least one absorbent in the inner region. This is in many cases advantageous with respect to the barrier function of the capsule wall. Such polymer structures are described for example in PCT/FR03/03465 or PCT/IB2004/004403. Their disclosure relating to this is hereby expressly incorporated in the description by reference.

Generally, however, according to a possible embodiment of the present invention, any other adsorbent polymer composition can also be used within the scope of the present invention, for example those described in U.S. Pat. No. 5,432,214, U.S. Pat. No. 5,911,937, U.S. Pat. No. 4,665,050, EP 0 432-438 or EP 0 400 460. Their corresponding disclosure is hereby expressly incorporated in the present description by reference.

As already mentioned, the adsorbent polymer materials used can further contain (conventional) organic or inorganic additives such as fibers, stabilizers, dyes, pigments, expanding agents, or combinations thereof or also other additives known from plastics processing. The amount of these additional substances is preferably reduced to a minimum. According to a preferred embodiment, the adsorbent polymer composition contains at least one adsorbing fiber component, in particular a superabsorber-fiber, e.g. of polyacrylate. According to another embodiment of the present invention, no wicking fibers are used in the polymer composition.

In a preferred embodiment, the dehydrating plastic does not possess any pronounced adhesion for pharmaceutical chemicals, in particular for particles of respirable size, so that when the container is used in an inhaler the entire contents of the capsule can be released. This ensures more accurate dosing, in particular of the respirable fine fraction of the pharmaceutical preparation.

Further information on possible polymer compositions or relating to the possible processing can be found in the aforementioned state of the art, in particular EP-A 0 599 690 and PCT/IB2004/004403.

In one embodiment the container wall can contain regions with varying composition of polymer/adsorbent. This applies for example in the case of capsules that comprise several different parts.

In some embodiments the capsule wall comprises at least two layers, an inner layer and at least one outer layer above it. Then the inner layer forms the direct wall of the cavity and is therefore in contact with the inhalation formulation. In this case one of the two layers can comprise the polymer composition with adsorbent, and the other layer comprises a pharmacologically neutral material. Sandwich structures are also possible, in which the outermost layer forms a first barrier to moisture, the middle layer comprises the polymer with adsorbent and the inner layer comprises a pharmacologically neutral material. Other layer sequences are possible. Suitable pharmacologically neutral materials are familiar to a person skilled in the art and comprise (in addition to the polymer compositions with at least one adsorbent used according to the invention) for example polymer compositions or plastics such as polyethylene, polypropylene or polystyrene.

The materials of the individual layers can then be selected so that they are not joined together integrally, or so that they are joined together integrally.

In yet another embodiment, the wall of the container is coated externally with the polymer composition containing the adsorbent, producing a continuous sealing surface. This variant has the advantage that on applying the absorbing, in particular the dehydrating layer, any joins that the walls of the cavities might have are sealed. This variant is especially suitable for two-part capsules.

The preferred capsule according to the invention is a two-part capsule. Such capsules preferably comprise two parts that telescope together, a capsule body (body) and a capsule cap (cap), which can be joined together so that a stable, enclosed cavity of a defined volume is formed, which contains the pharmaceutical preparation. The size of the capsule is such that it can be used e.g. in commonly available powder inhalers fitted with capsules.

In an especially preferred embodiment the cap and the body of the capsule are of cylindrical shape with a circular cross-section and closed convex ends of almost hemispherical shape.

In a preferred embodiment the capsule comprises a dehydrating plastic with a Shore hardness D from 60 to 80, preferably from 65 to 73.

Preferably said capsule is sufficiently stable to withstand a force of up to 10 N, more preferably up to 15 N along the longitudinal axis or the transverse axis. The advantage is that the capsule is more able to resist the stresses acting on the capsule during production, filling, packing, transportation etc.

In a preferred embodiment the cap and body of the capsule are of mutually similar, cylindrical shape, comprising a self-contained shell with in each case a closed and an open end. The cap and the capsule are of a shape and size such that the open end of the body can be inserted telescopically into the open end of the cap, so that the cap is joined firmly to the body.

In a special embodiment the cap and body are provided with closing devices, which are of advantage in temporary and/or final closure of the capsule.

In such an embodiment, there are raised dots on the inside surface of the cap and there are slightly larger dot-shaped depressions in the outside surface of the body, which are arranged so that during closure of the capsule the raised dots lock into the depressions. Alternatively, the raised dots can be formed on the outside surface of the body and the depressions on the inside surface of the cap. Arrangements are preferred in which the raised dots or the depressions are in each case arranged in a circle or spiral around the shell. Instead of raised dots and dot-shaped depressions, they can also form a continuous ring round the shell of the cap and of the body.

In one embodiment, one or more continuous circular projections are formed on the inside surface of the cap and on the outside surface of the body, so that when the capsule is in the closed state a projection of the cap is in each case next to a projection of the body.

In the embodiments with said circular depressions and/or projections these can be continuous or intermittent.

In another embodiment, projections are formed on the outside of the body near the open end and holes are formed in the cap near the open end, so that in the closed state of the capsule the projections of the body lock into the holes in the cap. The projections can in this case be of a form such that the cap can be opened at any time without damaging the capsule, or alternatively such that the capsule, once closed, can no longer be opened nondestructively.

In another embodiment, a bead is formed on the outside of the body, going round the body perpendicularly to the connecting axis between cap and body. The bead serves as a stop for the cap, when the latter is fitted over the body, to prevent the body being pushed through the cap. The region between the open end of the body and the bead corresponds to the region of the body over which the cap can be fitted. The bead is located on the body in such a way that the cap can be pushed far enough over the body to produce a firm closure between cap and body. This means that the bead is not positioned for example directly at the open end of the body. The side of the bead directed toward the open end of the body stands as a vertical edge on the outer wall of the body, so that the cap cannot be pushed beyond the bead during closure. The side of the bead directed toward the closed end of the body can be in the form of an almost right-angled edge or it can flatten out toward the closed end of the body. The design with an almost right-angled edge can be of advantage in the case of loose fitting of the cap in a cap holder, and the variant with a bead that flattens out can be of advantage in the case of a firm fit. The bead can be continuous or intermittent.

In a preferred embodiment, the bead flattens out continuously toward the closed end of the body and its side directed toward the open end of the body stands perpendicularly on the capsule body. The height of the edge thus formed is then such that the edge does not project beyond the capsule cap when the capsule is in the closed state, so that there is a flat transition from capsule cap to capsule body.

The thickness of the walls of the cap and of the body can vary in different regions. Thus, as a rule the wall thickness is greater in the rounded zones of the cap or of the body or at the point of the body where the bead is formed, than in the zones where the walls are straight. In one embodiment the walls of the cap and of the body have a thickness from 0.1 mm to 0.5 mm.

In another embodiment, pimples are formed on the outside of the capsule, and in another there are three or more ribs, running parallel to the longitudinal axis of the capsule. The advantage of these arrangements is that the capsule can be removed from a capsule holder, as used for example in the powder inhalers mentioned above, without being damaged or opened. The ribs or pimples can cover the entire outside of the capsule or just a portion thereof. Alternatively they can be formed only on the cap or only in the region of the body that is visible from outside when the capsule is in the closed state. The ribs run parallel to the longitudinal axis of the capsule and lock the capsule vertically in said capsule holder. In the case of a circular cross-section of the capsule, the ribs are preferably arranged so that the cross-section of the capsule does not have rotational symmetry about the central axis. In such an embodiment, the ribs can be formed only in the region of the body that is visible with the capsule in the closed state. Such an embodiment prevents jamming of the capsule in a capsule holder.

In one embodiment without a bead, but with ribs on the portion of the body that is visible in the closed state of the cap, the ribs are formed so that the ends of the ribs directed toward the open end of the body perform the function of the bead, namely to serve as a stop for the cap when fitting the cap and body together.

In another embodiment the shells of the cap and of the body describe a hollow cylinder of circular, oval, triangular, tetragonal, hexagonal, octagonal or polygonal cross-section, in each case with the top open and the bottom closed. The closed bottom can be flat or convex. The angular embodiments have, for example, the advantage that they save space in storage.

In one embodiment the elongation of the capsule (distance from the closed end of the body to the closed end of the cap in relation to the diameter with the capsule closed) is greater than 1, in another embodiment the elongation is equal to 1 and in yet another embodiment the elongation is less than 1. The latter has the advantage that the body has a larger opening for filling. In one of the embodiments with an elongation equal to 1 the cap and body are of a form such that the closed capsule is of spherical shape, which can be advantageous for automatic loading of an inhaler with the capsule from a reservoir.

In order to achieve a better seal between cap and body with the filled capsule in the closed state, the join between cap and body can be sealed by welding, gluing or banding, so that permeability to water vapor decreases considerably. Welding of cap and body is preferred. Alternatively the entire cap can be coated with a continuous protective film. The latter can be made of the material that is preferred according to the invention. In this case the capsule itself can be made of a material that does not contain an adsorbent.

In another preferred embodiment, the gap can be sealed with a filler. Suitable fillers for said filling of the gap are pharmaceutically permitted filler materials, for example Eudragit, a class of polymer based on methacrylic acid and methacrylates. Said filler can be introduced into the gap as a solution or suspension in a suitable, preferably highly volatile, solvent.

Such capsules are suitable containers of the type according to the invention for inhalers, for example those known by the tradenames HandiHaler®, Spinhaler®, Rotahaler®, Aerolizer®, Flowcaps®, Turbospin®, AIR DPI®, Orbital® and/or are described in DE 33 45 722, EP 0 591 136, DE 43 18 455, WO 91/02558, FR-A-2 146 202, U.S. Pat. No. 4,069,819, EP 666085, U.S. Pat. No. 3,991,761, WO99/45987.

A preferred invention relates to an assembly of an inhaler for the inhalation of pulverulent medicinal products and the capsule according to the invention, in particular a two-part capsule of cylindrical shape with rounded ends, the inhaler being characterized by a) an upward-opening, cup-shaped bottom part, which has two opposite windows in its shell and has a first hinge element at the edge of the opening, b) a plate, which covers the opening of the bottom part and has a second hinge element, c) an inhalation chamber for receiving the capsule, which is formed perpendicularly to the plane of the plate on the side of the plate directed toward the bottom part and on which a head is provided, which is movable against a spring, the head being provided with two ground needles, d) a top part with a mouthpiece and a third hinge element, and e) a lid, which has a fourth hinge element, the hinge elements of the bottom part, of the plate, of the top part and of the lid being joined together.

Preferably it is an inhaler of the HandiHaler® type.

Another aspect of the present invention relates to a secondary packaging, containing at least one capsule as described here, the secondary packaging preferably being moistureproof and more preferably having, at least partially, aluminum foil (e.g. a blister).

Yet another aspect of the present invention relates to the drying of small amounts of inhalable pharmaceutical formulations. Thus, it has been found that the capsules according to the invention not only are able to prevent or delay the entry of moisture into the capsules, so that the capsules provide a method by which inhalable pharmaceutical formulations can be kept dry, but also that with the capsules according to the invention, inhalable pharmaceutical formulations can be dried to low residual moisture. Pharmaceutical formulations in an amount of up to 50 mg, preferably up to 30 mg, especially preferably up to 15 and quite especially preferably up to 10 mg, are especially suitable. The moisture level can represent e.g. up to 5 wt. % of the formulation. The figures given relate to capsules of size 3. These and other suitable capsule sizes are described in more detail in the examples.

Finally, another aspect of the present invention relates to the use of a polymer composition as described herein (with at least one adsorbent) for the production of a capsule as described herein.

### EXAMPLES

Capsules of size 3 were produced as follows:

The adsorbent-polymer composition contained 67 wt. % high-density polyethylene (HDPE), 3 wt. % EVA (elastomer) and 30 wt. % of a synthetic zeolite (molecular sieve 4A, max. particle size 20 μm). First a premix of HDPE and EVA was prepared manually. This was blended with the synthetic zeolite in a twin-screw extruder. The compound was converted to granules. Unless the granules are to undergo further processing immediately, they can be sealed for storage in welded aluminum bags, to exclude moisture. The granules were processed by injection molding to capsules of size 3, setting MFI (melt flow index) of about 18 and using a 0.6 mm injection channel. According to a second variant the adsorbent polymer composition contained 66 wt. % high-density polyethylene (HDPE), 3 wt. % EVA (elastomer), 30 wt. % of a synthetic zeolite (molecular sieve 4A, max. particle size 20 μm) and additionally 1 wt. % of a synthetic polyacrylate superabsorber fiber.

The capsules obtained were filled with 5.5 mg of a water-sensitive formulation and then the top and bottom parts of the capsule were welded together.

The capsules thus produced were stored open, i.e. without additional secondary packaging, at a temperature of 40° C. and at 75% relative humidity.

After various times, the inhalable fraction of the particles (proportion by weight of particles with an aerodynamic size of less than 5 μm) was measured on discharge with a powder inhaler. The powder inhaler used was a device of the HandiHaler® type (WO94/28958), and the inhalable fraction was measured by means of a cascade impactor at a flow rate of 39 l/min.

As a result it was found that the in-use stability of the formulation in capsules comprising conventional material without addition of molecular sieve could be prolonged from 1 day by at least 9 days. The “in-use stability” is to be understood to mean the period between removal of the capsule from the packaging (e.g. the blister) and use of the capsule in the inhalation maneuver. During this period the capsule must display adequate stability, in order to protect the active substance that it contains e.g. against moisture. This is particularly important in the case of multidose inhalers.

The capsules according to the invention are preferably of the following size:

a) Length of capsule bodies: 22.2±0.46 mm; 20.22±0.46 mm; 20.98±0.46 mm; 18.4±0.46 mm; 16.61±0.46 mm; 15.27±0.46 mm; 13.59±0.46 mm; 12.19±0.46 mm; 9.3±0.46 mm.
b) Length of capsule cap: 12.95±0.46 mm; 11.74±0.46 mm; 11.99±0.46 mm; 10.72±0.46 mm; 9.78±0.46 mm; 8.94±0.46 mm; 8.08±0.46 mm; 7.21±0.46 mm; 6.2±0.46 mm.
c) Outside diameter of capsule bodies: 9.55 mm; 8.18 mm; 7.36 mm; 7.34 mm; 6.63 mm; 6.07 mm; 5.57 mm; 5.05 mm; 4.68 mm.
d) Outside diameter of capsule caps: 9.91 mm; 8.53 mm; 7.66 mm; 7.64 mm; 6.91 mm; 6.35 mm; 5.83 mm; 5.32 mm; 4.91 mm.
e) Total length of the closed capsule: 26.1±0.3 mm; 23.3±0.3 mm; 24.2±0.3 mm; 21.7±0.3 mm; 19.4±0.3 mm; 18.0±0.3 mm; 15.9±0.3 mm; 14.3±0.3 mm; 11.1±0.3 mm.
f) Capsule volumes: 1.37 ml; 0.95 ml; 0.78 ml; 0.50 ml; 0.37 ml; 0.30 ml; 0.21 ml; 0.13 ml.

The invention will be explained in more detail below on the basis of drawings:

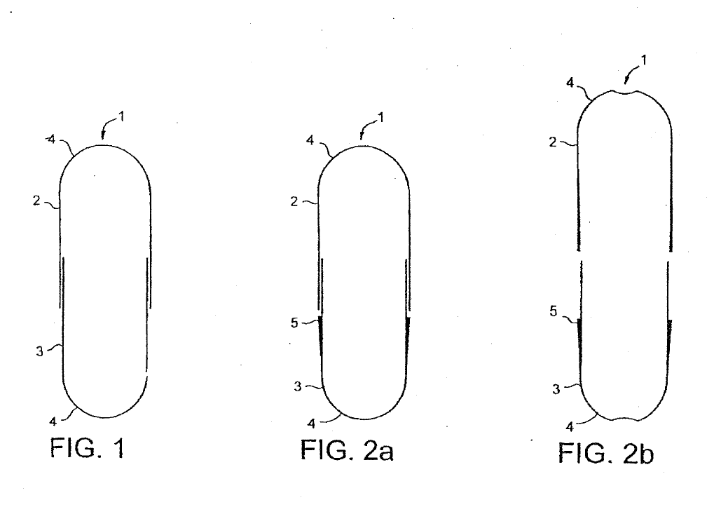

FIG. 1 shows the simplest embodiment of the capsule according to the invention 1 in cross-section. The capsule 1 comprises the cap 2 and the body 3, which are inserted telescopically in one another. Cap 2 and body 3 are of the same shape and they each have a convex bottom 4.

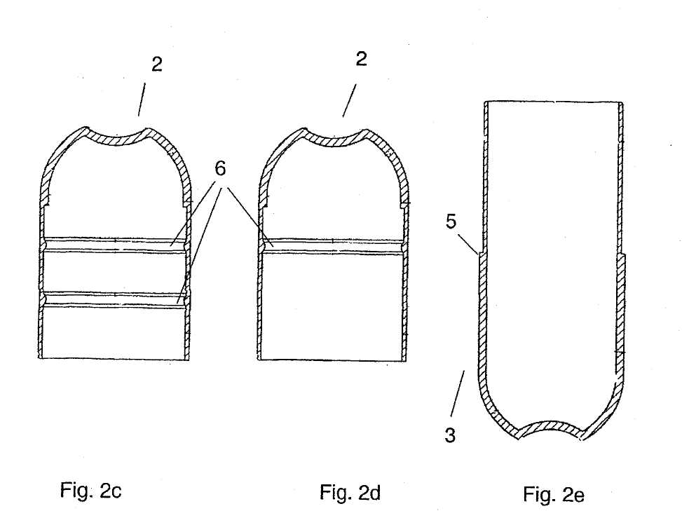

FIG. 2a shows, in cross-section, an embodiment in which a bead 5 is formed on the body 3 of capsule 1, said bead tapering toward the closed end of the body. The side of bead 5 directed toward the open end of the body is almost perpendicular on the body. The edge thus formed limits the region of the body over which cap 2 can be pushed telescopically.

Another embodiment is depicted in FIG. 2b. The cross-section shows that this embodiment differs from that shown in FIG. 2a in that the wall thickness of cap 2 or of body 3 is not of identical thickness over the entire region, but varies over individual partial regions. In addition, the convex bottoms 4 of the cap and of the body each have a concave indentation at the vertex. FIGS. 2c and 2d show capsule caps with projections on the inside surface 6. FIG. 2e shows a further embodiment of the capsule body, which differs from that shown in FIGS. 1, 2a and 2b with respect to the wall thickness.

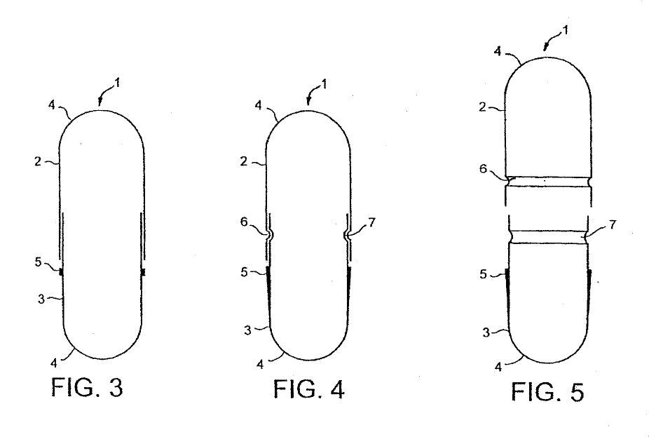

FIG. 3 shows an embodiment in which bead 5 is almost at right angles on the body both at the top and at the bottom of the body.

The embodiment in FIG. 4 represents a further development of the embodiment in FIG. 2a, in which a circular depression or projection 6 or 7 is formed in cap 2 or body 3 for better closure of capsule 1.

FIG. 5 shows a front view of the embodiment shown in cross-section in FIG. 4.

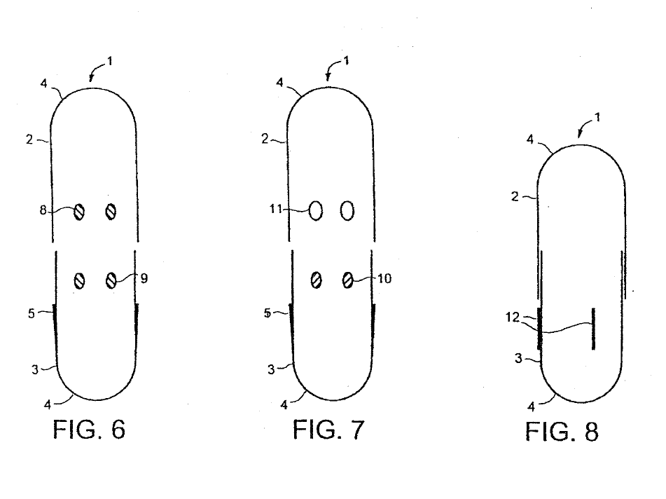

FIG. 6 shows another variant of the invention with dot-shaped depressions 8 and 9 in front view.

FIG. 7 shows a variant of capsule 1, with projections 10 formed on body 3 near the open end, and holes 11 formed in cap 2 near the open end, so that during closure of the capsule the projections 10 lock into the holes 11.

FIG. 8 shows an external view of an embodiment of capsule 1, in which ribs 12 are formed on body 3.

The capsules as shown in the drawings can be made, in one embodiment, from the material according to the invention, in particular from polyethylene with dehydrating agent. In another embodiment the capsule is made from a polymer and this material is then sprayed over it. In another embodiment the capsule is then banded or laminated with metal foil, e.g. aluminum foil.

1. A capsule comprising at least one cavity enclosed by a wall, wherein at least one portion of the wall comprises a polymer composition that contains at least one adsorbent.

2. The capsule as claimed in claim 1, further comprising at least two cylindrical partial elements that can be inserted telescopically in one another, wherein said capsule is an integral component of a ready-to-use powder inhaler.

3. The capsule as claimed in claim 1, characterized in that the at least one portion of the wall comprises primarily, substantially or completely the polymer composition with at least one adsorbent.

4. The capsule as claimed in claim 1, characterized in that the polymer composition contains comprises at least one thermoplastic material.

5. The capsule as claimed in claim 4, characterized in that the thermoplastic material comprises at least one polyolefin.

6. The capsule as claimed in claim 1, characterized in that the absorbent is in the form of particles, with a maximum particle size of less than 50 μm.

7. The capsule as claimed in claim 1, characterized in that the thickness of the capsule wall in at least a section thereof is between about 0.05 and 2 mm.

8. The capsule as claimed in claim 1, characterized in that the ratio of the particle size of the absorbent to the thickness of the capsule wall is between 0.01 and 0.2.

9. The capsule as claimed in claim 4, characterized in that the thermoplastic material is selected from the group consisting of polystyrene, polyolefins, in particular polyethylene or polypropylene; polyacrylates, polymethacrylates; polyimides, polycarbonates, polyethersulfones, polyamides, polyesters; and polyvinyl chlorides; styrene-butadiene rubber (SBR); styrene-ethylene-butadiene-styrene copolymers (SEBS); butyl rubber; ethylene-propylene rubber (EPR); ethylene-propylene-diene-monomer rubber (EPDM); ethylene-vinyl acetate copolymer (EVA); ethylene-acrylate or butadiene-acrylonitrile; maleic anhydride modified polymers and copolymers; and graft copolymers.

10. The capsule as claimed in claim 1, characterized in that the adsorbent is selected from the group consisting of silica gels, zeolites, drying clays or clays that absorb moisture or water, aluminosilicates such as zeolites or bentonites, molecular sieves, activated carbon, alkaline-earth oxides, calcium sulfate and mixtures thereof.

11. The capsule as claimed in claim 1, characterized in that the adsorbent is selected from the group consisting of silica gel, aluminosilicates, such as bentonites, molecular sieves and calcium sulfate.

12. The capsule as claimed in claim 1, characterized in that the polymer composition is selected from the group consisting of a polymer from a single monomer, a copolymer from two or more monomers, a mixture of two or more polymers each from a single monomer, a mixture of two or more copolymers, and a mixture of at least one polymer from a single monomer and at least one copolymer.

13. The capsule as claimed in claim 4, characterized in that the polymer composition or the thermoplastic material comprises a mixture of at least one polymer from a single monomer and at least one copolymer, wherein at least one copolymer of the thermoplastic material has a monomer unit in common with the polymer.

14. The capsule as claimed in claim 1, characterized in that the capsule wall comprises at least one migration zone on at least one surface of the capsule wall and an inner region, where the maximum concentration of the adsorbent within the migration zone is at least twice as high as the maximum concentration of the adsorbent in the inner region.

15. The capsule as claimed in claim 1, characterized in that the capsule wall is formed homogeneously from the polymer composition with the at least one adsorbent and presents a monolithic structure.

16. The capsule as claimed in claim 1, characterized in that the capsule wall is formed from two or more layers.

17. The capsule as claimed in claim 16, characterized in that the capsule wall has an inner layer and at least one outer layer, with the inner layer forming the immediate wall of the capsule cavity and with one of the two layers comprising the polymer composition with the at least one adsorbent and the other layer comprising a pharmacologically neutral material.

18. The capsule as claimed in claim 16, characterized in that the capsule wall comprises a sandwich structure, where the outermost layer forms a barrier to moisture, the middle layer comprises the polymer composition with the at least one adsorbent and the inner layer comprises a pharmacologically neutral material.

19. The capsule as claimed in claim 1, characterized in that the polymer composition further comprises plasticizers, stabilizers, colorants or pigments.

20. The capsule as claimed in claim 1, characterized in that the capsule is a two-part capsule, which in the closed state is in the shape of a cylinder with rounded closed ends, which is formed by a capsule body, which is inserted telescopically into a capsule cap.

21. The capsule as claimed in claim 1, characterized in that the capsule comprises a capsule body and a capsule cap, the walls of which have a thickness of 0.1 mm to 0.5 mm.

22. The capsule as claimed in claim 21, characterized in that the join between body and cap is sealed by welding.

23. (canceled)

24. An inhaler containing at least the capsule of claim 1.

25. Secondary packaging, containing at least one capsule as claimed in claim 1.

26. A method of drying premetered amounts of inhalable powders with a residual moisture of up to 5 wt. % in amounts of up to 50 mg, characterized in that the powder is stored in the capsule as claimed in claim 1.

27. The method as claimed in claim 26, characterized in that the capsule is enclosed in a secondary packaging.

28. The method as claimed in claim 27, characterized in that the secondary packaging is moistureproof.

29. The method as claimed in claim 27, characterized in that the secondary packaging is a blister, which has at least partially an aluminum foil.

30. The capsule of claim 1 utilized as packaging for inhalation formulations.
